(19) United States
(12) Patent Application Publication
(10) Pub. No.: US 2019/0048416 A1
(43) Pub. Date: Feb. 14, 2019

(54) TREATMENT OF FIBROTIC CONDITIONS

(71) Applicant: GILLIES MCINDOE RESEARCH INSTITUTE, Wellington (NZ)

(72) Inventors: Swee Thong Tan, Wellington (NZ); Paul Frank Davis, Wellington (NZ); Tinte Itinteang, Wellington (NZ)

(21) Appl. No.: 15/779,216

(22) PCT Filed: Nov. 30, 2016
(86) PCT No.: PCT/NZ2016/050187

§ 371 Date: May 25, 2018

Related U.S. Application Data
(60) Provisional application No. 62/260,953, filed on Nov. 30, 2015.

(51) Int. Cl. C12Q 1/6881

(52) U.S. Cl. CPC C12Q 1/6881; C12Q 2600/118; C12Q 2600/158

(57) ABSTRACT

The present invention provides a novel approach to the diagnosis, treatment and management of a fibrotic lesion by providing compositions and methods for the identification and specific targeting of the embryonic stem cell populations known to be associated with a fibrotic lesion, by modulation of the Renin-Angiotensin System.

### TECHNICAL FIELD

The present invention provides novel approaches to the treatment of fibrotic conditions. In particular, the present invention provides identification of embryonic stem cell populations shown to be associated with fibrotic lesions, which provide a unique therapeutic target in the treatment of fibrotic conditions.

### BACKGROUND OF THE INVENTION

Fibrosis is the formation of excess fibrous connective tissue in an organ or tissue in a reparative or reactive process. This can be a reactive, benign, or pathological state. In response to injury, this is called scarring, and if fibrosis arises from a single cell line, this is called a fibroma. Physiologically, fibrosis acts to deposit connective tissue, which can obliterate the architecture and function of the underlying organ or tissue. Fibrosis can be used to describe the pathological state of excess deposition of fibrous tissue, as well as the process of connective tissue deposition in healing.

Fibrosis is similar to the process of scarring, in that both involve stimulated cells laying down connective tissue, including collagen and glycosaminoglycans. Immune cells including macrophages, as well as any damaged tissue between surfaces called interstitium, release TGF beta. There are numerous reasons for this, including inflammation of the nearby tissue, or a generalised inflammatory state, with increased circulating mediators. TGF beta stimulates the proliferation and activation of fibroblasts, which synthesise and subsequently secrete connective tissue.

Fibrosis can occur in many tissues within the body, typically as a result of inflammation or damage, and examples include lungs (pulmonary fibrosis, cystic fibrosis, idiopathic pulmonary fibrosis), liver (cirrhosis), heart (atrial fibrosis, endomyocardial fibrosis, old myocardial infarction), knee, shoulder and other joints (arthrofibrosis), intestine (Crohn's Disease, abdominal adhesions), hands and fingers (Dupuytren's contracture), skin (keloid, nephrogenic systemic fibrosis), soft tissue of the mediastinum (mediastinal fibrosis), bone marrow (myelofibrosis), penis (Peyronie's disease), skin/lungs (scleroderma/systemic sclerosis) and kidney.

Keloid scar (KS) is characterised by abnormal, excessive and ongoing deposition of collagen type 3-rich tissue (Thompson 2004), which develop over time in response to an injury to the skin (Berman & Bieley 1995). The excessive response may result from trivial injury, and usually presents as an overgrowth of raised scar tissue which continues to enlarge over many years (Berman & Bieley 1995). Keloid scar eventually stabilises, and may exhibit some central regression (Muir 1990). However, keloid scar has also been known to occur spontaneously without documented wounding, or appear several years after an injury (Berman & Bieley 1995). Unlike normal and hypertrophic scars (HTS), keloid scar extends into the adjacent normal skin beyond the boundaries of the original wound, and clinically resembles a benign tumour (Berman & Bieley 1995). While HTS develops soon after wounding, keloid scar may not be evident for several months following the initial injury (English & Shenefelt 1999).

The incidence of keloid is higher in darker-skinned individuals (Thompson 2004), affects males and females equally, and has a familial tendency (Berman & Bieley 1995).

Current treatment for keloid scar is unsatisfactory and includes repeated intralesional corticosteroid injections, or peri-lesional excision with concomitant intraoperative steroid injections, pressure dressing, and application of silicone gel, either singly or in combination, with varying success (Al-Attar 2006). Surgical excision alone is typically futile, with recurrence rates around 72% (Furtado 2012). Surgical excision followed by immediate adjuvant therapy (Al-Attar 2006) has been used in difficult cases. However, radiotherapy-induced malignancy remains a concern, especially in younger patients (Botwood 1999).

Keloid scar consists of several cell types with fibroblasts and myofibroblast being the most predominant (James 1980). Stem cells exhibiting clonogenicity, multipotency and self-renewal abilities have also been demonstrated in keloid scar, and these contribute to the pathological environment required for benign tumour-like cell growth (Zhang 2009). Keloid scar has been shown to express the embryonic stem cell (ESC) markers, OCT4 and SSEA-4 (Zhang 2009), while another recent report has demonstrated the presence of CD20-expressing lymphoid cells which are organised into distinctive aggregates known as keloid-associated lymphoid tissue (KALT; Bagabir 2012). However, what remains to be determined is the exact localisation of this primitive population within keloid scar.

The present invention has established expression of the ESC markers OCT4, critical for cell cycle progression of ESCs; SOX2, that support the expression ESC genes; pSTAT3, involved in a wide range of cellular processes; and NANOG, a pluripotency-associated transcription factor, in keloid scar.

### SUMMARY OF THE INVENTION

The inventions described and claimed herein have many attributes and embodiments including, but not limited to, those set forth or described or referenced in this Summary of the Invention. It is not intended to be all-inclusive and the inventions described and claimed herein are not limited to or by the features or embodiments identified in this Summary of the Invention, which is included for purposes of illustration only and not restriction.

Applicants have identified discrete populations of embryonic stem cells that are associated with an extensive range of fibrotic conditions. Accordingly, identification of these embryonic stem populations provides a novel approach to the management of fibrotic conditions, as well as in prognostic, diagnostic and follow-up applications. In addition, the Applicants have surprisingly demonstrated that these embryonic stem cells express markers associated with key regulatory systems including, for example, the Renin-Angiotensin System (RAS) including the Pro/Renin Receptor System (PRRS) and the associated bypass pathways. This insight provides a novel target and unique therapeutic opportunity in the management of fibrotic conditions by employing established and/or novel drugs that specifically target these regulatory pathways in an attempt to eradicate, or arrest growth, proliferation and/or differentiation of embryonic stem cell populations responsible for the formation of fibrotic lesions.

Accordingly, in one aspect of the present invention there is provided a method for preventing, treating, or managing a fibrotic condition in a patient in need thereof, the method comprising administering a therapeutic agent to the patient in an amount sufficient to selectively eradicate, or inhibit the growth, proliferation and/or differentiation of embryonic stem cells associated with a fibrotic lesion, wherein the embryonic stem cells are characterised by (i) the expression of one or more embryonic stem cell biomarkers, and (ii) the expression of one or more biomarkers associated with the Renin-Angiotensin System.

In yet another aspect of the present invention there is provided a method for preventing, treating, or managing a fibrotic condition in a patient in need thereof, the method comprising administering a therapeutic agent to the patient in an amount sufficient to selectively eradicate, or inhibit the growth, proliferation and/or differentiation of embryonic stem cells associated with a fibrotic lesion, wherein the embryonic stem cells are characterised by (i) the expression of one or more embryonic stem cell biomarkers, and (ii) the expression of one or more biomarkers associated with the Renin-Angiotensin System, and wherein the fibrotic condition is selected from the group consisting of liver fibrosis, kidney fibrosis, lung fibrosis, hypertrophic scars, keloid scars, Dupuytren's contracture and desmoid tumours.

In another aspect of the present invention there is provided a method for preventing, treating, or managing a fibrotic condition in a patient in need thereof, the method comprising administering a therapeutic agent to the patient in an amount sufficient to selectively eradicate, or inhibit the growth, proliferation and/or differentiation of embryonic stem cells associated with the fibrotic lesion, wherein the embryonic stem cells are characterised by (i) the expression of one or more stem cell biomarkers selected from the group consisting of Cripto, ABCG2, Alkaline Phosphatase/ALPL, CD9, FGF-4, GDF-3, Integrin alpha 6/CD49f, Integrin beta 1/CD29, NANOG, OCT-3/4, Podocalyxin, SOX2, SSEA-3, SSEA-4, STAT3, SSEA-1, FoxD3, DPPA5/ESG1, Rex-1/ZFP42, DPPA4, LIN-28A, UTF1, Lefty-A, Lefty-1, TBX3, ESGP, TRA-1-60(R), TRA-1-81, 5T4, TBX2, ZIC3, CD30/TNFRSF8, KLF5, c-Myc, GCNF/NR6A1, SUZ12, Smad2, CDX2, TROP-2, CD117/c-kit, LIN-41, Integrin alpha 6 beta 4, THAP11, Smad2/3, TBX5, TEX19, Oct-4A, TEX19.1, DPPA2, Activin RIB/ALK-4, Activin RIIB, FGF-5, GBX2, Stella/Dppa3, DNMT3B, F-box protein 15/FBXO15, LIN-28B, Integrin alpha 6 beta 1, KLF4, ERR beta/NR3B2, EpCAM/TROP1, TERT, CHD1, Cbx2, c-Maf, L1TD1, and (ii) the expression of one or more biomarkers associated with the Renin-Angiotensin System.

In yet another aspect of the present invention there is provided a method for preventing, treating, or managing a fibrotic condition in a patient in need thereof, the method comprising administering a therapeutic agent to the patient in an amount sufficient to selectively eradicate, or inhibit the growth, proliferation and/or differentiation of embryonic stem cells associated with a fibrotic lesion, wherein the embryonic stem cells are characterised by (i) the expression of one or more embryonic stem cell biomarker selected from the group consisting of OCT4, SOX2, NANOG and PSTAT3, and (ii) the expression of one or more biomarkers associated with the Renin-Angiotensin System selected from the group consisting of Renin Receptor (RR), Angiotensin II Receptor 2, a soluble form of the Renin Receptor (sRR) and a soluble form of Angiotensin Converting Enzyme (ACE).

In yet a further aspect of the present invention there is provided a method for preventing, treating, or managing a fibrotic condition in a patient in need thereof, the method comprising administering a therapeutic agent(s) to the patient in an amount sufficient to selectively eradicate or, inhibit the growth, proliferation and/or differentiation of embryonic stem cells associated with a fibrotic lesion, wherein the embryonic stem cells are characterised by (i) the expression of one or more stem cell biomarkers selected from the group consisting of Oct-4, SOX2, NANOG and PSTAT3, and (ii) the expression of one or more biomarkers associated with the Renin-Angiotensin System selected from the group consisting of Renin Receptor, Angiotensin II Receptor 2 and a secreted form of the Renin Receptor, and wherein the therapeutic agent is selected from the group consisting of Direct Renin Inhibitors (DRIs), Angiotensin-Converting Enzyme Inhibitors (ACEIs), Angiotensin Receptor Blockers (ARBs), Beta-Blockers, Cyclo-oxygenase 2 Inhibitors, Chymase Inhibitors, Inhibitors of Cathepsin B, Cathepsin D and Cathepsin G, Calcium, Vitamin D, and Calcium Channel Blockers.

In yet another aspect of the present invention there is provided a method for determining the presence or absence of a fibrotic lesion in a subject, the method comprising:

(i) detecting and/or measuring the levels of embryonic stem cells present in a biological sample obtained from the subject using biomarker expression analysis;

(ii) comparing the levels of the embryonic stem cells obtained from the biological sample against the level from a control population;

wherein, an increased level in the embryonic stem cells obtained from the biological sample relative to the control population is diagnostic that the subject has, or is predisposed to developing, a fibrotic condition.

In another aspect of the present invention there is provided a method for determining presence or absence of a fibrotic condition in a subject, the method comprising:

(i) detecting and/or measuring the level of embryonic stem cells and/or components of the Renin-Angiotensin system in a biological sample obtained from the subject using biomarker expression analysis;

(ii) comparing the level of the embryonic stem cells and/or components of the Renin-Angiotensin system obtained from the biological sample against the level from a control population,

wherein, an increased level in the embryonic stem cells and/or components of the Renin-Angiotensin system obtained from the biological sample relative to the control population is diagnostic that the subject has, or is predisposed to developing, a fibrotic condition, and

(iii) administering a prophylactic or therapeutic regime to the subject who has, or is predisposed to developing, the fibrotic condition.

In another aspect of the present invention there is provided a pharmaceutical composition for use in a method for treatment of a fibrotic condition, wherein the pharmaceutical composition comprises a therapeutic agent sufficient to selectively eradicate or, inhibit the growth, proliferation and/or differentiation of embryonic stem cells associated with a fibrotic lesion, and wherein the method comprises administering the therapeutic agent to a patient with a fibrotic lesion.

In another aspect of the present invention there is provided a kit or article of manufacture for use in the treatment of a fibrotic condition, the kit comprising a therapeutic agent sufficient to selectively eradicate, or inhibit the growth, proliferation and/or differentiation of embryonic stem cells associated with a fibrotic condition, together with instructions for how to administer a therapeutic dose to the subject.

### BRIEF DESCRIPTION OF THE FIGURES

FIG. 1 shows immunofluorescent immunohistochemical (IHC) stained images of representative keloid scar (KS) tissues demonstrating the expression of OCT4 (A, green, white arrows) on the endothelium expressing vWF (A, red). The co-expression of both proteins on the same cells is portrayed as orange (A). The same endothelium, highlighted by the expression of CD34 (B-D, green), also expressed SOX2 (B, red, white arrows), pSTAT3 (C, red, white arrows) and NANOG (D, red, white arrows). Cell nuclei are counterstained with DAPI.

FIG. 2 shows DAB IHC staining of representative KS tissues confirming the endothelium, expressing vWF (A & B, red), is surrounded by both CD20+ (A, brown) and CD3+ (B, brown) cells. Original magnification: 400×.

FIG. 3 shows Log₁₀ relative expression of CD163, CD34, NANOG, OCT4 and STAT3 over the HPRT1 housekeeper (n=6).

FIG. 4 shows in situ hybridization (ISH) staining on representative KS samples showing the expression of OCT4 (A, red, black arrows), SOX2 (B, red, black arrows), STAT3 (C, red, black arrows) and NANOG (D, red, black arrows). Cell nuclei are counterstained with haematoxylin. Original magnification: 1000×.

FIG. 5 Immunofluorescent IHC staining of human seminoma (positive control) samples for OCT4 (A, green), SOX2 (B, red), pSTAT3 (C, red) and NANOG (D, red). Cell nuclei are counterstained with DAPI (A-D, blue). Original magnification: 400×.

FIG. 6 shows DAB IHC staining of human tonsillar (positive control) samples for vWF (A & B, red) and either CD20 (A, brown) or CD3 (B, brown). Cell nuclei are counterstained with haematoxylin (A & B, blue). Original magnification: 400×.

FIG. 7 shows ISH staining of human seminoma (positive control) samples for OCT4 (A, red), SOX2 (B, red), STAT3 (C, red) and NANOG (D, red). Cell nuclei are counterstained with haematoxylin (A-D, blue). Original magnification: 1000×.

FIG. 8 shows co-localisation of Renin Receptor on the endothelium that also expresses the embryonic stem cells derived from keloid scars.

FIG. 9 shows the main pathways associated with the RAS. ACE: Angiotensin Converting Enzyme; ACEIs: Angiotensin Converting Enzyme inhibitors; Cox2i: Cox2 inhibitors; β-blockers: Beta-Blockers; ATIIR2: Angiotensin II Receptor 2; ATIIR1: Angiotensin II Receptor 1; (Pro)-RR: Pro(Renin) Receptors [also called Renin Receptor (RR)]; Vit D: Vitamin D; XX: major blockades; ++: major promoting steps.

FIG. 10 shows the combined pathways associated with the RAS. ACE: Angiotensin Converting Enzyme; ACEI: Angiotensin Converting Enzyme Inhibitors; Cox2i: Cox2 inhibitors; β-blockers: Beta-Blockers; ATIIR2: Angiotensin II Receptor 2; ATIIR1: Angiotensin II Receptor 1; (Pro)-RR: Pro(Renin) Receptors [also called Renin Receptor (RR)]; Vit D: Vitamin D; XX: major blockades; X: minor blockades; ++: major promoting steps; +: minor blocking steps.

### SELECTED DEFINITIONS

Unless defined otherwise, all technical and scientific terms used herein have the same meaning as commonly understood to one of ordinary skill in the art to which the inventions belong. Although any assays, methods, devices and materials similar or equivalent to those described herein can be used in the practice or testing of the invention, various assays, methods, devices and materials are now described.

It is intended that reference to a range of numbers disclosed herein (for example 1 to 10) also incorporates reference to all related numbers within that range (for example, 1, 1.1, 2, 3, 3.9, 4, 5, 6, 6.5, 7, 8, 9 and 10) and also any range of rational numbers within that range (for example 2 to 8, 1.5 to 5.5 and 3.1 to 4.7) and, therefore, all sub-ranges of all ranges expressly disclosed herein are expressly disclosed. These are only examples of what is specifically intended and all possible combinations of numerical values between the lowest value and the highest value enumerated are to be considered to be expressly stated in this application in a similar manner.

As used in this specification, the words “comprises”, “comprising”, and similar words, are not to be interpreted in an exclusive or exhaustive sense. In other words, they are intended to mean “including, but not limited to”.

As used herein, the term “antibodies” refer to molecules that contain an antigen binding site, e.g., immunoglobulins. Immunoglobulin molecules can be of any type (e.g., IgG, IgE, IgM, IgD, IgA and IgY), class (e.g., IgG1, IgG2, IgG3, IgG4, IgA1 and IgA2) or subclass. Antibodies include, but are not limited to, monoclonal antibodies, polyclonal antibodies, multispecific antibodies, human antibodies, humanised antibodies, murine antibodies, camelised antibodies, chimeric antibodies, single domain antibodies, single chain Fvs (scFv), single chain antibodies, Fab fragments, F(ab′) fragments, disulfide-linked Fvs (sdFv), and anti-idiotopic (anti-Id) antibodies (including, e.g., anti-Id antibodies to antibodies of the invention), and epitope-binding fragments of any of the above.

As used herein, the term “effective amount” refers to the amount of a therapy that is sufficient to result in the prevention of the development, recurrence, or onset of a fibrotic condition and one or more symptoms thereof, to enhance or improve the prophylactic effect(s) of another therapy, reduce the severity, the duration of fibrosis, ameliorate one or more symptoms of fibrosis, prevent the advancement of fibrosis, cause regression of fibrosis, and/or enhance or improve the therapeutic effect(s) of another therapy.

As used herein, the terms “manage”, “managing”, and “management” in the context of the administration of a therapy to a subject refer to the beneficial effects that a subject derives from a therapy (e.g., a prophylactic or therapeutic agent) or a combination of therapies, while not resulting in a cure of fibrosis. In certain examples, a subject is administered one or more therapies (e.g., one or more prophylactic or therapeutic agents) to “manage” fibrosis so as to prevent the progression or worsening of the condition.

As used herein, the terms “prevent”, “preventing” and “prevention” in the context of the administration of a therapy to a subject refers to the prevention or inhibition of the recurrence, onset, and/or development of fibrosis or a symptom thereof in a subject resulting from the administration of a therapy (e.g., a prophylactic or therapeutic agent), or a combination of therapies (e.g., a combination of prophylactic or therapeutic agents).

As used herein, the term “marker” or “biomarker” in the context of a tissue means any antigen, molecule or other chemical or biological entity that is specifically found in or on a tissue that it is desired to be identified or identified in or on a particular tissue affected by a disease or disorder, for example fibrosis. In specific examples, the marker is a cell surface antigen that is differentially or preferentially expressed by specific cell types. In specific examples, the marker is a nuclear antigen that is differentially or preferentially expressed by specific cell types. In specific examples the marker is an intracellular antigen that is differentially or preferentially expressed by specific cell types.

As used herein, the term “prophylactic agent” refers to any molecule, compound, and/or substance that is used for the purpose of preventing fibrosis. Examples of prophylactic agents include, but are not limited to, proteins, immunoglobulins (e.g., multi-specific Igs, single chain Igs, Ig fragments, polyclonal antibodies and their fragments, monoclonal antibodies and their fragments), antibody conjugates or antibody fragment conjugates, peptides (e.g., peptide receptors, selectins), binding proteins, proliferation based therapy, and small molecule drugs.

As used herein, the term “therapeutic agent” refers to any molecule, compound, and/or substance that is used for the purpose of treating and/or managing a disease or disorder. Examples of therapeutic agents include, but are not limited to, proteins, immunoglobulins (e.g., multi-specific Igs, single chain Igs, Ig fragments, polyclonal antibodies and their fragments, monoclonal antibodies and their fragments), peptides (e.g., peptide receptors, selectins), binding proteins, biologics, proliferation-based therapy agents, hormonal agents, radioimmunotherapies, targeted agents, epigenetic therapies, differentiation therapies, biological agents, and small molecule drugs.

As used herein, the terms “therapies” and “therapy” can refer to any method(s), composition(s), and/or agent(s) that can be used in the prevention, treatment and/or management of fibrosis or one or more symptoms thereof.

As used herein, the terms “treat”, “treatment” and “treating” in the context of the administration of a therapy to a subject refer to the reduction or inhibition of the progression and/or duration of fibrosis, the reduction or amelioration of the severity of fibrosis, and/or the amelioration of one or more symptoms thereof resulting from the administration of one or more therapies.

The term “sample” or “biological sample” as used herein means any sample taken or derived from a subject. Such a sample may be obtained from a subject, or may be obtained from biological materials intended to be provided to the subject. For example, a sample may be obtained from blood being assessed, for example, to investigate fibrosis in a subject. Included are samples taken or derived from any subjects such as from normal healthy subjects and/or healthy subjects for whom it is useful to understand their fibrosis status. Preferred samples are biological fluid samples. The term “biological fluid sample” as used herein refers to a sample of bodily fluid obtained for the purpose of, for example, diagnosis, prognosis, classification or evaluation of a subject of interest, such as a patient. The sample may be any sample known in the art in which embryonic stem cells may be detected. Included are any body fluids such as a whole blood sample, plasma, serum, ovarian follicular fluid sample, seminal fluid sample, cerebrospinal fluid, saliva, sputum, urine, pleural effusions, interstitial fluid, synovial fluid, lymph, tears, for example, although whole blood sample, plasma and serum are particularly suited for use in this invention. In addition, one of skill in the art would realise that certain body fluid samples would be more readily analysed following a fractionation or purification procedure, for example, separation of whole blood into serum or plasma components.

The term “purified” as used herein does not require absolute purity. Purified refers in one embodiment to at least 90%, or 95%, or 98%, or 99% homogeneity of, to provide an example, of a polypeptide or antibody in a sample.

The term “subject” as used herein is preferably a mammal and includes human, and non-human mammals such as cats, dogs, horses, cows, sheep, deer, mice, rats, primates (including gorillas, rhesus monkeys and chimpanzees), possums and other domestic farm or zoo animals. Thus, the assays, methods and kits described herein have application to both human and non-human animals, in particular, and without limitation, humans, primates, farm animals including cattle, sheep, goats, pigs, deer, alpacas, llamas, buffalo, companion and/or pure bred animals including cats, dogs and horses. Preferred subjects are humans, and most preferably “patients” who as used herein refer to living humans who may receive or are receiving medical care or assessment for a disease or condition. Further, while a subject is preferably a living organism, the invention described herein may be used in post-mortem analysis as well.

The term “ELISA” as used herein means an enzyme linked immunosorbent assay, a type of competitive binding assay comprising antibodies and a detectable label used to quantitate the amount of an analyte in a sample.

The term “capture antibody” as used herein means an antibody which is typically immobilized on a solid support such as a plate, bead or tube, and which antibody binds to and captures analyte(s) of interest, for example membrane bound markers associated with an embryonic stem cell population.

The term “detection antibody” as used herein means an antibody comprising a detectable label that binds to an analyte(s) of interest. The label may be detected using routine detection means for a quantitative, semi-quantitative or qualitative measure of the analyte(s) of interest, for example membrane bound markers associated with an embryonic stem cell population.

As used herein, the term “relating to the presence or amount” of an analyte reflects that assay signals are typically related to the presence or amount of an analyte through the use of a standard curve calculated using known concentrations of the analyte of interest. As the term is used herein, an assay is “configured to detect” an analyte if an assay can generate a detectable signal indicative of the presence or amount of a physiologically relevant concentration of the analyte. Typically, an analyte is measured in a sample.

A level “higher” or “lower” than a control, or a “change” or “deviation” from a control (level) in one embodiment is statistically significant. A higher level, lower level, deviation from, or change from a control level or mean or historical control level can be considered to exist if the level differs from the control level by about 5% or more, by about 10% or more, by about 20% or more, or by about 50% or more compared to the control level. Statistically significant may alternatively be calculated as P≤0.05. Higher levels, lower levels, deviation, and changes can also be determined by recourse to assay reference limits or reference intervals. These can be calculated from intuitive assessment or non-parametric methods. Overall, these methods may calculate the 0.025, and 0.975 fractiles as 0.025* (n+1) and 0.975 (n+1). Such methods are well known in the art. Presence of a marker absent in a control may be seen as a higher level, deviation or change. Absence of a marker present in a control may be seen as a lower level, deviation or change.

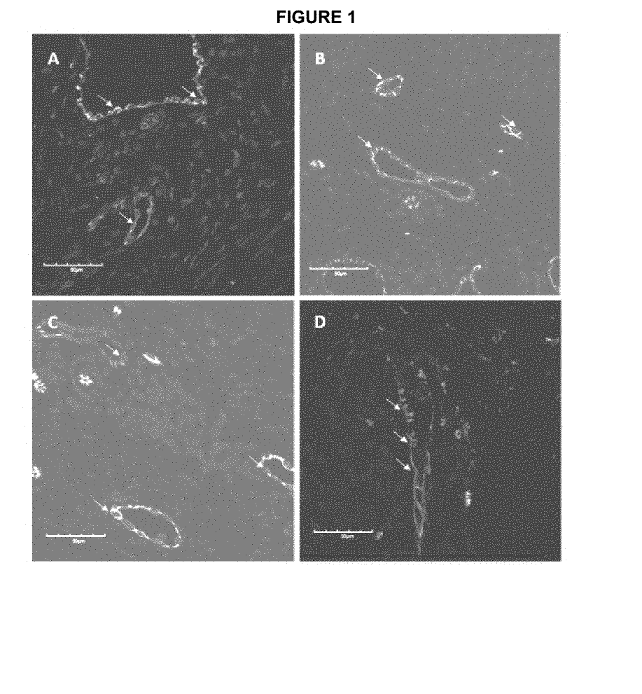

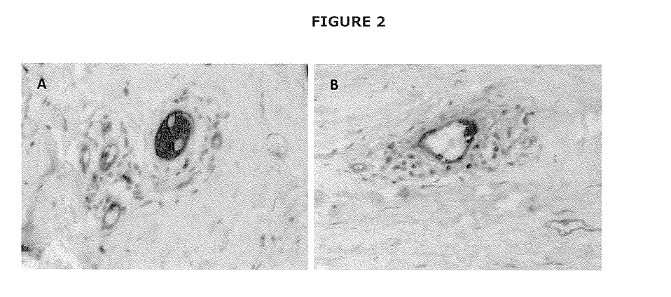

As used herein, the term “Renin-Angiotensin System (RAS)” or “Renin-Angiotensin-Aldosterone System (RAAS)” is a hormone system that regulates blood pressure and fluid balance. The wider pathway associated with RAS also includes the Pro/Renin Receptor System (PRRS) and the associated bypass pathways. By way of example, refer to FIGS. 1 and 2. There are a number of known drugs which target the RAS including PRRS, as described in more detail below.

### DETAILED DESCRIPTION

The present invention is predicated on the surprising and unexpected discovery that discrete populations of embryonic stem cells are associated with fibrotic lesions associated with, for example, liver fibrosis, kidney fibrosis, lung fibrosis, hypertrophic scars, keloid scar, Dupuytren's contracture and desmoid tumours. The embryonic stem cell populations associated with these lesions may be characterised by unique biomarker expression profiles that allow for the specific identification and diagnosis of certain fibrotic conditions. Refer to, for example, FIGS. 1-7.

Expression of embryonic-like stem cell populations in keloid associated lymphoid aggregates may be crucial in the development and growth of keloid scars.

Importantly, Applicants have demonstrated that the keloid scar associated primitive cell population is located immediately deep to the epidermis of the skin, and that these populations of embryonic-like stem cells may play a key role in the pathogenesis of keloid scars.

Accordingly, in one aspect of the present invention there is provided a method for preventing, treating, or managing a fibrotic condition in a patient in need thereof, the method comprising administering a therapeutic agent to the patient in an amount sufficient to selectively eliminate or inhibit the growth, proliferation and/or differentiation of embryonic stem cells associated with a fibrotic lesion, wherein the embryonic stem cells are characterised by the expression of one or more embryonic stem cell biomarkers.

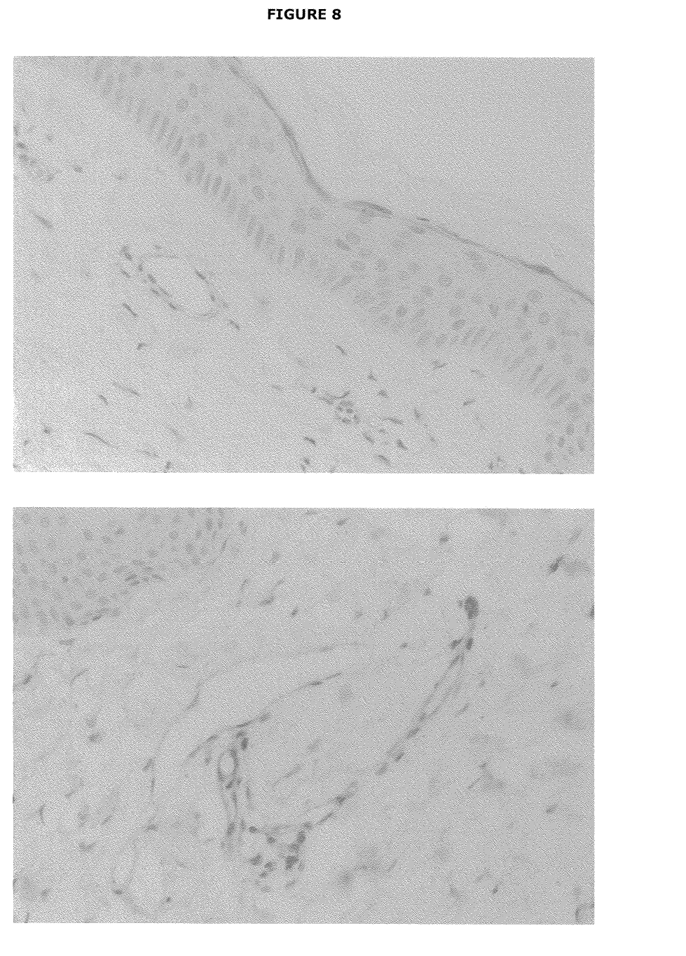

Further, it has also been revealed by the Applicants that these embryonic-like stem cell populations express key components of the the Renin-Agiotensin System (RAS), including the Renin Receptor (RR). In reference to FIG. 8, Applicants demonstrate co-expression of Renin Receptor by the embryonic stem cell populations associated with keloid scars. These embryonic stem cell populations are characterised by, for example, the expression of OCT4, SOX2, PSTAT3 and NANOG. Further, it is possible that these embryonic stem cells also express Angiotensin II Receptor 2 (ATIIR2), as well as soluble forms of the Renin Receptor (sRR) and Angiotensin Converting Enzyme (ACE). Accordingly, the expression of the components of RAS by these embryonic stem cell populations provides a novel and unique therapeutic approach by targeting the embryonic stem cells associated with various fibrotic lesions from the extensive array of drugs that target RAS such as, Angiotensin-Converting Enzyme Inhibitors (ACEis), Angiotensin Receptor Blockers (ARBs), Direct Renin Inhibitors (DRIs), Beta-Blockers, Cyclo-oxygenase 2 Inhibitors, Chymase Inhibitors, Inhibitors of Cathepsin B, Cathepsin D and Cathepsin G, Calcium Supplements, Vitamin D and Calcium Channel Blockers.

In addition, the present invention also contemplates indirect inhibitors of the RAS (e.g., Calcium Channel Blockers).

Accordingly, in one aspect of the present invention there is provided a method for preventing, treating, or managing a fibrotic condition in a patient in need thereof, the method comprising administering a therapeutic agent to the patient in an amount sufficient to selectively eliminate or inhibit the growth, proliferation and/or differentiation of embryonic stem cells associated with a fibrotic lesion, wherein the embryonic stem cells are characterised by (i) the expression of one or more embryonic stem cell biomarkers, and (ii) the expression of one or more biomarkers associated with the Renin-Angiotensin System.

In another aspect of the present invention there is provided a method for preventing, treating, or managing a fibrotic condition in a patient in need thereof, the method comprising administering a therapeutic agent to the patient in an amount sufficient to selectively inhibit the growth, proliferation and/or differentiation of embryonic stem cells associated with a fibrotic lesion, wherein the embryonic stem cells are characterised by (i) the expression of one or more embryonic stem cell biomarkers, and (ii) the expression of one or more biomarkers associated with the Renin-Angiotensin System, and wherein the fibrotic condition is selected from the group consisting of liver fibrosis, kidney fibrosis, lung fibrosis, hypertrophic scars, keloid scar, Dupuytren's contracture and desmoid tumours.

In another aspect of the present invention there is provided a method for preventing, treating, or managing a fibrotic condition in a patient in need thereof, the method comprising administering a therapeutic agent to the patient in an amount sufficient to selectively inhibit the growth, proliferation and/or differentiation of embryonic stem cells associated with a fibrotic lesion, wherein the embryonic stem cells are characterised by (i) the expression of one or more embryonic stem cell biomarkers, and (ii) the expression of one or more biomarkers associated with the Renin-Angiotensin System, and wherein the fibrotic condition is keloid scar.

In one example, the one or more embryonic stem cell markers is selected from the group consisting of Cripto, ABCG2, Alkaline Phosphatase/ALPL, CD9, FGF-4, GDF-3, Integrin alpha 6/CD49f, Integrin beta 1/CD29, Nanog, Oct-3/4, Podocalyxin, SOX2, SSEA-3, SSEA-4, STAT3, SSEA-1, FoxD3, DPPA5/ESG1, Rex-1/ZFP42, DPPA4, LIN-28A, UTF1, Lefty-A, Lefty-1, TBX3, ESGP, TRA-1-60(R), TRA-1-81, 5T4, TBX2, ZIC3, CD30/TNFRSF8, KLF5, c-Myc, GCNF/NR6A1, SUZ12, Smad2, CDX2, TROP-2, CD117/c-kit, LIN-41, Integrin alpha 6 beta 4, THAP11, Smad2/3, TBX5, TEX19, Oct-4A, TEX19.1, DPPA2, Activin RIB/ALK-4, Activin RIIB, FGF-5, GBX2, Stella/Dppa3, DNMT3B, F-box protein 15/FBXO15, LIN-28B, Integrin alpha 6 beta 1, KLF4, ERR beta/NR3B2, EpCAM/TROP1, TERT, CHD1, Cbx2, c-Maf and L1TD1. In another example, the one or more embryonic stem cell biomarkers consists in OCT4, SOX2, NANOG and PSTAT3. In yet another example, the one or more biomarkers associated with the RAS is selected from the group consisting of Renin Receptor, Angiotensin II Renin Receptor, a soluble form of the Renin Receptor and a soluble form of Angiotensin Converting Enzyme.

In another aspect of the present invention there is provided a method for preventing, treating, or managing a fibrotic condition in a patient in need thereof, the method comprising administering a therapeutic agent to the patient in an amount sufficient to selectively eliminate, or inhibit the growth, proliferation and/or differentiation of embryonic stem cells associated with a fibrotic lesion, wherein the embryonic stem cells are characterised by (i) the expression of one or more stem cell biomarker selected from the group consisting of Cripto, ABCG2, Alkaline Phosphatase/ALPL, CD9, FGF-4, GDF-3, Integrin alpha 6/CD49f, Integrin beta 1/CD29, NANOG, OCT3/4, Podocalyxin, SOX2, SSEA-3, SSEA-4, STAT3, SSEA-1, FoxD3, DPPA5/ESG1, Rex-1/ZFP42, DPPA4, LIN-28A, UTF1, Lefty-A, Lefty-1, TBX3, ESGP, TRA-1-60(R), TRA-1-81, 5T4, TBX2, ZIC3, CD30/TNFRSF8, KLF5, c-Myc, GCNF/NR6A1, SUZ12, Smad2, CDX2, TROP-2, CD117/c-kit, LIN-41, Integrin alpha 6 beta 4, THAP11, Smad2/3, TBX5, TEX19, Oct-4A, TEX19.1, DPPA2, Activin RIB/ALK-4, Activin RIIB, FGF-5, GBX2, Stella/Dppa3, DNMT3B, F-box protein 15/FBXO15, LIN-28B, Integrin alpha 6 beta 1, KLF4, ERR beta/NR3B2, EpCAM/TROP1, TERT, CHD1, Cbx2, c-Maf and L1TD1, and (ii) the expression of one or more biomarkers associated with the Renin-Angiotensin System.

In yet another aspect of the present invention there is provided a method for preventing, treating, or managing a fibrotic condition in a patient in need thereof, the method comprising administering a therapeutic agent to the patient in an amount sufficient to selectively eliminate, or inhibit the growth, proliferation and/or differentiation of embryonic stem cells associated with a fibrotic lesion, wherein the embryonic stem cells are characterised by (i) the expression of one or more stem cell marker selected from the group consisting of OCT4, SOX2, NANOG and PSTAT3, and (ii) the expression of one or more biomarkers associated with the Renin-Angiotensin System selected from the group consisting of Renin Receptor, Angiotensin II Receptor 2, a soluble form of the Renin Receptor and a soluble form of Angiotensin Converting Enzyme.

In yet a further aspect of the present invention there is provided a method for preventing, treating, or managing a fibrotic condition in a patient in need thereof, the method comprising administering a therapeutic agent to the patient in an amount sufficient to selectively eradicate, or inhibit the growth, proliferation and/or differentiation of embryonic stem cells associated with a fibrotic lesion, wherein the embryonic stem cells are characterised by (i) the expression of one or more stem cell biomarker selected from the group consisting of OCT4, SOX2, NANOG and PSTAT3, and (ii) the expression of Renin Receptor, Angiotensin II Receptor 2 and/or a secreted form of Renin Receptor, and wherein the therapeutic agent is selected from the group consisting of Direct Renin Inhibitors (DRIs), ACEis, ARBs, Beta-Blockers, Cyclo-oxygenase 2 Inhibitors, Chymase Inhibitors, Inhibitors of Cathepsin B, Cathepsin D and Cathepsin G, Calcium, Vitamin D, and Calcium Channel Blockers.

In one example, the embryonic stem cells are characterised by the expression of SOX2, OCT4, PSTAT3 and NANOG. These cells are said to have a marker expression profile: SOX2⁺Oct-4⁺PSTAT3⁺NANOG⁺.

In a related example, the embryonic stem cells are embryonic stem cells associated with a keloid scar and are characterised by the marker expression profile CD44⁺SOX2⁺OCT4⁺NANOG⁺. In a further example, the embryonic stem cells associated with a keloid scar are characterised by the marker expression profile CD44⁺SOX2⁺OCT4⁺NANOG⁺CD34⁻. In yet a further example, the embryonic stem cells associated with a keloid scar are characterised by the marker expression profile CD44⁺SOX2⁺OCT4⁺NANOG⁺CD34⁻p63⁻EMA⁻.

The embryonic stem cells may co-express with other embryonic stem cell markers, lymphatic cell markers, or any combination thereof.

The present invention provides compositions and methods related to identifying and targeting the growth and proliferation of embryonic stem cells as the cause of fibrotic lesions. By specifically targeting these embryonic stem cells, it is assumed that the lesion potential is significantly diminished, thereby leading to enhanced therapeutic outcomes.

The embryonic stem cells may be associated with a variety of fibrotic conditions, including but not limited to, liver fibrosis, kidney fibrosis, lung fibrosis, hypertrophic scars, keloid scar, Dupuytren's contracture and desmoid tumours.

The present invention provides methods for preventing, treating, and/or managing fibrotic conditions including lesion formation, the method comprising administering to a subject in need thereof a course of therapy that stabilises, reduces, or eradicate the embryonic stem cell population. In certain examples, the stabilisation, reduction, or elimination of the embryonic stem cell population is achieved by administering a therapy that targets the growth and proliferation of the stem cells.

Surprisingly, Applicants demonstrate that the embryonic stem cell populations identified in the methods according to the present invention co-express components of RAS in multiple different lesion types. By way of illustration only, the Applicants demonstrate co-expression of the RR and embryonic stem cells (FIG. 8).

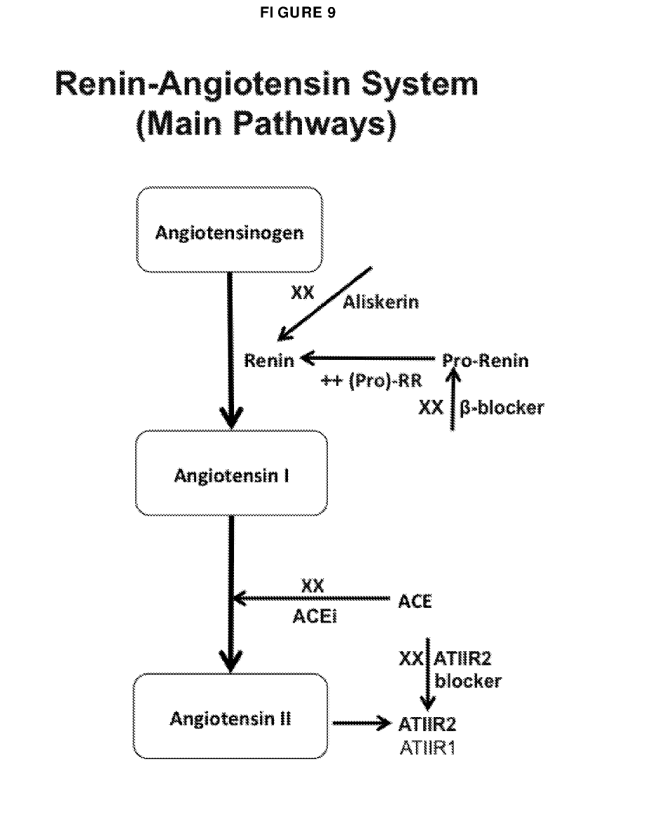

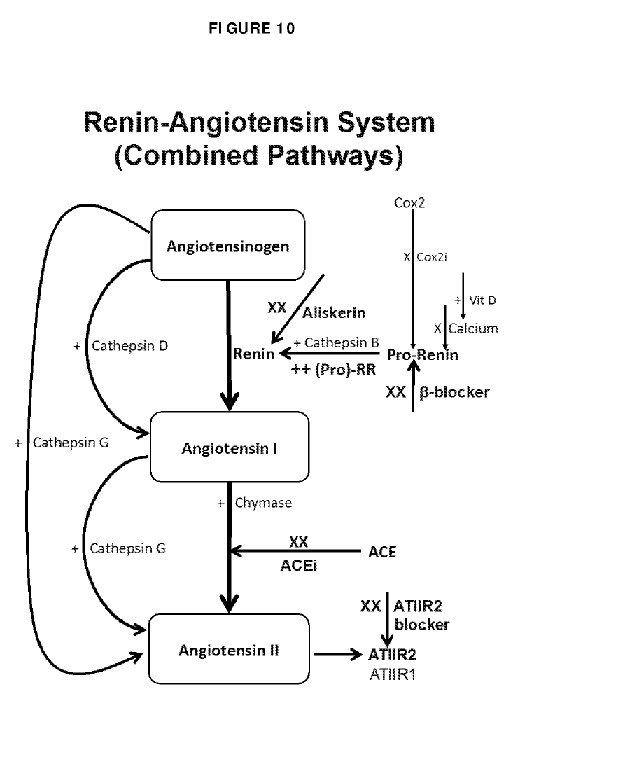

Accordingly, therapy that targets the growth and proliferation of embryonic stem cell populations comprises administering a therapeutic agent that selectively targets components of the RAS and/or Pro/Renin Receptor Systems (PRRS) expressed by the stem cells. FIGS. 9 and 10 show the types of inhibitors/drugs that target these systems, useful in accordance with the compositions and methods according to the present invention.

Examples of known therapeutics that target the Renin-Angiotensin System include, but are not limited to, ACEIs, ARBs, DRIs, Beta-Blockers, Cyclo-oxygenase 2 Inhibitors, Inhibitors of Cathepsin B, Cathepsin D and Cathepsin G, Calcium Channel Blockers, Calcium Supplements and Vitamin D.

Examples of ACEIs include, but are not limited to, Benazepril (Lotesin), Captopril (Capoten), Cilazipril, Enalapril (Vasotec, Renitec), Fosinopril (Monopril), Lisinopril (Lisodur, Lopril, Novatec, Prinivil, Zestril), Moexipril, Perindopril (Coversay, Aceon), Quinapril (Accupril), Ramipril (Altace, Tritace, Ramace, Ramiwin), Trandolapril, Delapril, Zofenopril and Imidapril.

Examples of ARBs include, but are not limited to, Losartan, Irbesartan, Candesartan, Eprosartan, Olmesartan, Telmisartan, PD123319 and Valsartan.

Examples of Beta-Blockers include, but are not limited to, Acebutolol (Sectral), Atenolol (Tenormin), Betaxolol (Betoptic), Bisoprolol (Cardicor, Emcor, Zebeta), Carteolol (Teoptic), Carvedilol (Coreg, Eucardic), Celiprolol (Celectol), Labetalol (Trandate), Levobunolol (Betagan), Metipranolol (Metipranolol Minims), Metoprolol (Betaloc, Lopresor, Lopressor, Toprol XL), Nadolol (Corgard), Nebivolol (Bystolic, Nebilet), Oxprenolol (Trasicor), Pindolol (Visken), Propranolol (Inderal LA), Sotalol (Beta-Cardone, Sotacor), and Timolol (Betim, Nyogel, Timoptol).

Examples of Cyclo-oxygenase 2 Inhibitors include, but are not limited to, Celecoxib, Nepafenac, Ibuprofen (Dolgesic), Indomethacin, Sulindac, Xanthohumol, Meclofenamate Sodium, Meloxicam, Rofecoxib, Bromfenac Sodium, Ibuprofen Lysine, Ketorolac (Ketorolac tromethamine), Diclofenac Sodium, Etodolac, Ketoprofen, Naproxen Sodium, Piroxicam, Acemetacin, Phenacetin, Tolfenamic Acid, Nimesulide, Flunixin Meglumin, Aspirin, Bufexamac, Niflumic acid, Licofelone, Oxaprozin, Lornoxicam, Lumiracoxib, Zaltoprofen, Ampiroxicam, Valdecoxib, Nabumetone, Mefenamic Acid, Carprofen, Amfenac Sodium monohydrate, Asaraldehyde and Suprofen.

Examples of Chymase Inhibitors include, but are not limited to, TY-51469 (2-[4-(5-fluoro-3-methylbenzo[b]thiophen-2-yl)sulfonamido-3-methanesulfonyl-phenyl]thiazole-4-carboxylic acid), Eglin C, CI, SUN13834, Chymostatin, TJK002 a benzimidazole inhibitor, ONO-WH-236, Amblyomma americanum tick serine protease inhibitor 6 (AamS6), N-tosyl-L-phenylalanyl chloromethyl ketone (TPCK), Alpha-aminoalkylphosphonate diaryl esters, Serine protease inhibitor A3 (serpinA3), Squamous cell carcinoma antigen (SCCA-2), Bortezomib (Velcade), R05066852 and 17beta-estradiol.

Examples of Cathepsin B Inhibitors include, but are not limited to, Cystatin B, Cystatin C, Cysteine peptidase inhibitor E64, [Pt(dmba)(aza-N1)(dmso)] complex 1 (a potential anti-tumoral drug with lower IC50 than cisplatin in several tumoral cell lines), 2,3,7,8-Tetrachlorodibenzo-p-dioxin (TCDD), CA-074Me, Lipidated CtsB inhibitor incorporated into the envelope of a liposomal nanocarrier (LNC-NS-629), Proanthocyanidin (PA) and ahpatinin Ac (1) and ahpatinin Pr (2).

Examples of Cathepsin D Inhibitors include, but are not limited to, non-peptidic acylguanidine inhibitors of Cathepsin D, Pepstatin A, Bm-Aspin, SlPI, Via, RNAi-Rab27A and Solanum lycopersicum aspartic protease inhibitor (SLAPI).

Examples of Cathepsin G Inhibitors include, but are not limited to, WFDC12, Phenylmethylsulfonyl fluoride (PMSF), Ecotin, SerpinB1, SerpinA3, CeEI, or Caesalpinia echinata elastase inhibitor, SLPI (secretory leukocyte protease inhibitor), Alpha1-Antitrypsin (AAT), Bauhinia bauhinoides cruzipain inhibitor, Alpha-Aminoalkylphosphonate diaryl esters, Greglin, [2-[3-[[(1-benzoyl-4-piperidinyl)methylamino]carbonyl]-2-naphthalenyl]-1-(1-naphthalenyl)-2-oxoethyl]-phosphonic acid (KPA), Lympho-Epithelial Kazal-Type-related Inhibitor (LEKTI), Trappin-2 A62L, SV-66, SCGI, Bortezomib, Human monocyte/neutrophil elastase inhibitor (MNEI), a 42-kDa serpin protein and Anti-leukoproteinase (ALP).

Examples of Calcium Channel Blockers include, but are not limited to, Dihydropyridine Calcium Channel Blockers, Phenylalkylamine Calcium Channel Blockers, Benzothiazepine Calcium Channel Blockers, Non-Selective Calcium Channel Blockers, as well as “Other” Calcium Channel blockers.

Examples of Dihydropyridine Calcium Channel Blockers include, but are not limited to, Amlodipine (Norvasc), Aranidipine (Sapresta), Azelnidipine (Calblock), Barnidipine (HypoCa), Benidipine (Coniel), Cilnidipine (Atelec, Cinalong, Siscard) Not available in US, Clevidipine (Cleviprex), Isradipine (DynaCirc, Prescal), Efonidipine (Landel), Felodipine (Plendil), Lacidipine (Motens, Lacipil), Lercanidipine (Zanidip), Manidipine (Calslot, Madipine), Nicardipine (Cardene, Carden SR), Nifedipine (Procardia, Adalat), Nilvadipine (Nivadil), Nimodipine (Nimotop), Nisoldipine (Baymycard, Sular, Syscor), Nitrendipine (Cardif, Nitrepin, Baylotensin), Pranidipine (Acalas).

Examples of Phenylalkylamine Calcium Channel Blockers include, but are not limited to, Verapamil (Calan, Isoptin), Gallopamil and Fendiline.

Examples of Benzothiazepine Calcium Channel Blockers include, but are not limited to, Diltiazem (Cardizem) and Fendiline.

Examples of Non-Selective Calcium Channel Blockers include, but are not limited to, Mibefradil, Bepridil, Flunarizine, Fluspirilene and Fendiline.

Examples of other Calcium Channel Blockers include, but are not limited to, Gabapentin, Pregabalin and Ziconotide.

An example of DRIs includes, but is not limited to, Aliskiren.

In certain examples, the embryonic stem cells may be partially differentiated and committed toward a specific cell lineage associated with a particular fibrotic lesion.

The present invention also contemplates identification of embryonic stem cells as a means of diagnosing a fibrotic condition, by profiling expression of certain markers known to be associated with the embryonic stem cells.

Accordingly, in another aspect of the present invention there is provided a method for determining presence or absence of a fibrotic condition in a subject, the method comprising:

(i) detecting and/or measuring the levels of embryonic stem cells present in a biological sample obtained from the subject using biomarker expression analysis;

(ii) comparing the levels of the embryonic stem cells obtained from the biological sample against the embryonic stem cell level from a control population;

wherein, an increased level in the embryonic stem cells obtained from the biological sample relative to the control population is diagnostic that the subject has, or is predisposed to developing, a fibrotic lesion.

The present invention further contemplates identification of embryonic stem cells as a means of diagnosing a fibrotic condition, by profiling expression markers of the Renin Angiotensin system known to be associated with the embryonic stem cells.

Accordingly, in another aspect of the present invention there is provided a method for determining presence or absence of a fibrotic condition in a subject, the method comprising:

(i) detecting and/or measuring the level of embryonic stem cells and/or components of the Renin-Angiotensin system in a biological sample obtained from the subject using biomarker expression analysis;

(ii) comparing the level of the embryonic stem cells and/or components of the Renin-Angiotensin system obtained from the biological sample against the stem cell level from a control population,

wherein, an increased level in the embryonic stem cells obtained from the biological sample relative to the control population is diagnostic that the subject has, or is predisposed to developing, a fibrotic lesion, and

(iii) administering a prophylactic or therapeutic regime to the subject who has, or is predisposed to developing, a fibrotic lesion.

The present invention also contemplates pharmaceutical compositions comprising a therapeutic active or drug useful in the treatment of a fibrotic condition.

Accordingly, in another aspect of the present invention there is provided a pharmaceutical composition for use in a method for treatment of a fibrotic condition, wherein the pharmaceutical composition comprises a therapeutic agent(s) sufficient to selectively eradicate or, inhibit the growth, proliferation and/or differentiation of embryonic stem cells associated with a fibrotic lesion, and wherein the method comprises administering the therapeutic agent to a patient with a fibrotic condition.

In certain examples of the pharmaceutical compositions according to the present invention, the therapeutic agent is selected from the group consisting of Direct Renin Inhibitors (DRIs), Angiotensin-Converting Enzyme Inhibitors (ACEIs), Angiotensin Receptor Blockers (ARBs), Beta-Blockers, Cyclo-oxygenase 2 Inhibitors, Chymase Inhibitors, Inhibitors of Cathepsin B, Cathepsin D and Cathepsin G, Calcium, Vitamin D, and Calcium Channel Blockers.

The present invention also contemplates kits and articles of manufacture comprising a therapeutic active or drug useful in the treatment of a fibrotic condition.

Accordingly, in another aspect of the present invention there is provided a kit for use in the treatment of a fibrotic condition, the kit comprising a therapeutic agent sufficient to selectively eradicate, or inhibit the growth, proliferation and/or differentiation of embryonic stem cells associated with a fibrotic lesion, together with instructions for how to administer a therapeutic dose to the subject.

In certain examples of the kits according to the present invention, the therapeutic agent is selected from the group consisting of Direct Renin Inhibitors (DRIs), Angiotensin-Converting Enzyme Inhibitors (ACEIs), Angiotensin Receptor Blockers (ARBs), Beta-Blockers, Cyclo-oxygenase 2 Inhibitors, Chymase Inhibitors, Inhibitors of Cathepsin B, Cathepsin D and Cathepsin G, Calcium, Vitamin D, and Calcium Channel Blockers.

Further detail with respect to the present invention is presented under the following sub-headings.

### Renin-Angiotensin System

The Renin-Angiotensin System (RAS) is traditionally known to preserve fluid volume during periods of restricted dietary salt and also prevents ischaemia during acute volume loss. The main effector peptide of the RAS is angiotensin II (ATII). It induces vasoconstriction and sympathetic activation, raises aldosterone levels, and promotes renal salt and water retention via the angiotensin II receptor 1 (ATIIR1). Over the last few decades, the RAS has been a drug target of particular interest because of its involvement in cardiovascular disease (CVD) and renovascular disease. The CVD and renovascular disease can be understood as a continuum of risk factors, target organ damage, events, and mortality. Risk factors (such as hypertension, dyslipidemia, diabetes, and smoking) led to the development of target organ damage including atherosclerosis, left ventricular hypertrophy (LVH), and renal impairment. Target organ damage progressively worsens, leading ultimately to myocardial infarction (MI), heart failure (HF), end-stage renal disease (ESRD), stroke, or death.

ATII, the main effector peptide of the RAS, plays an active role during all stages of this continuum. The first step in the RAS cascade is the formation of angiotensin I (ATI) from the precursor angiotensinogen under the action of renin; early evidence for the importance of RAS in CVD came from the consistent finding that renin activity is predictive of the risk of cardiovascular (CV) events. ATI is then converted to ATII, the principal effector peptide of the RAS, by angiotensin-converting enzyme (ACE). In addition, ATII can be produced in tissues by enzymes such as chymase. This locally produced ATII is believed to mediate paracrine and autocrine functions. ATII acts via ATIIR1 and ATIIR2. Activation of ATIIR1 results in vasoconstriction, aldosterone and vasopressin secretion, sodium retention, and decreased renal perfusion. Hence, these receptors mediate the deleterious effects of ATII, including elevated blood pressure (BP) and cardiac and vascular remodelling. The effects of the ATII receptors have been less clearly defined because of the limited expression of these receptors in adults, because of their unconventional signalling pathways, and because many ATII-mediated actions are masked by opposing ATI-mediated effects. However, it is now recognised that ATIIR2 generally opposes the actions of ATIIR1, mediating various antiproliferative and anti-inflammatory effects and promoting tissue differentiation and regeneration and apoptosis.

Additional components of the RAS have been identified in the last decade, including bioactive angiotensin peptides, such as angiotensin III, angiotensin IV, and angiotensin-(1-7), the effects of which have not yet been fully elucidated for the CV and renal system.

The discovery of the renin receptor has shed further light on the biology of the RAS. Renin, simply considered until recently as the rate-limiting enzyme of RAS activation, has also turned out to be the ligand for a protein known as the renin/prorenin receptor that binds renin and prorenin about equally, regardless of their biologic activities. Prorenin, which represents 70% to 90% of total circulating renin, when bound to the receptor induces an increase in the catalytic efficiency of angiotensinogen conversion to ATI, which contributes to local production of ATII and its systemic levels, as well as binding of renin/prorenin to the renin/prorenin receptor, exerting physiologic effects that are independent of ATII, including activation of intracellular signal pathways, enhanced synthesis of DNA, and stimulation of the release of plasminogen activator inhibitor 1, collagen 1, fibronectin, and transforming growth factor β-1.6

There are a number of known drugs which target the RAS. The two major classes of drugs that target the RAS are the angiotensin-converting enzyme (ACE) inhibitors and the selective ATI receptor blockers (ARBs). Although both of these drug classes target ATII, the differences in their mechanisms of action have implications for their effects on other pathways and receptors that may have therapeutic implications. Both ACEIs and ARBs are effective antihypertensive agents that have been shown to reduce the risk of cardiovascular and renal events.

Direct inhibition of renin, the most proximal aspect of the RAS, became clinically feasible from 2007 with the introduction of Aliskiren. This latter drug has been shown to be efficacious for the management of hypertension. Combined therapy of direct renin-inhibitors with ACEIs or ARBs has been tested in some clinical situations as congestive HF and proteinuria with diverse results.

RAS drugs include, but are not limited to, Angiotensin-Converting Enzyme Inhibitors (ACEIs), Angiotensin Receptor Blockers (ARBs), Direct Renin Inhibitors (DRIs), Beta-Blockers, Cyclo-oxygenase 2 Inhibitors, Chymase Inhibitors, Cathepsin Inhibitors including Cathepsin B Inhibitors, Cathepsin D Inhibitors and Cathepsin G Inhibitors, Calcium Channel Blockers, Calcium Supplements and Vitamin D, as described above.

### Articles of Manufacture

The present invention also encompasses a finished packaged and labelled pharmaceutical product(s). This article of manufacture includes the appropriate unit dosage form in an appropriate vessel or container such as a glass vial or other container that is hermetically sealed. The pharmaceutical product may contain, for example, a prophylactic or therapeutic agent in a unit dosage form in a first container, and in a second container, sterile water for injection. Alternatively, the unit dosage form may be a solid suitable for oral, transdermal, intranasal, or topical delivery.

In a specific example, the unit dosage form is suitable for intravenous, intramuscular, intranasal, oral, topical or subcutaneous delivery. Thus, the invention encompasses solutions, preferably sterile, suitable for each delivery route.

In some examples, the pharmaceutical product is a prophylactic and/or therapeutic agent disclosed herein. In some examples, the pharmaceutical product is a composition comprising a prophylactic and/or therapeutic agent and a pharmaceutically acceptable carrier or excipient. In a specific example, the pharmaceutical composition is in a form for an appropriate route of administration. Such routes include, without limitation, oral, topical, parenteral, sublingual, rectal, vaginal, ocular, intradermal, intratumoural, intracerebral, intrathecal, and intranasal routes.

As with any pharmaceutical product, the packaging material and container are designed to protect the stability of the product during storage and shipment. Further, the products of the invention include instructions for use or other informational material that advise the physician, technician or patient on how to appropriately prevent or treat the disease or disorder in question. In other words, the article of manufacture includes instruction means indicating or suggesting a dosing regimen including, but not limited to, actual doses, the frequency of administration, the duration of administration monitoring procedures for cell counts, lymphocyte counts, neutrophil counts, and other monitoring information.

Specifically, the invention provides an article of manufacture comprising packaging material, such as a box, bottle, tube, vial, container, sprayer, insufflator, intravenous (i.v.) bag, envelope and the like; and at least one unit dosage form of a pharmaceutical agent contained within said packaging material, wherein said pharmaceutical agent comprises a prophylactic or therapeutic agent, and wherein said packaging material includes instruction means which indicate that said agent can be used to prevent, manage, treat, and/or ameliorate one or more symptoms associated with fibrosis, or one or more symptoms thereof by administering specific doses and using specific dosing regimens as described herein.

In certain examples, the article of manufacture include labeled antibodies that selectively or specifically bind to embryonic stem cells. As such, the article contains a method to adjust the dosages used in the treatment regimens, and to monitor the efficacy of the regimens.

The present invention provides that the adverse effects that may be reduced or avoided by the methods of the invention are indicated in informational material enclosed in an article of manufacture for use in preventing, treating and/or managing a fibrotic condition. Adverse effects that may be reduced or avoided by the methods of the invention include, but are not limited to, vital sign abnormalities (fever, tachycardia, bardycardia, hypertension, hypotension), haematological events (anaemia, lymphopenia, leukopenia, thrombocytopenia), headache, chills, dizziness, nausea, asthenia, back pain, chest pain (chest pressure), diarrohea, myalgia, pain, pruritus, psoriasis, rhinitis, sweating, injection site reaction, and vasodilation.

Further, the information material enclosed in an article of manufacture for use in preventing, treating and/or managing a fibrotic lesion can indicate that foreign proteins may also result in allergic reactions, including anaphylaxis, or cytosine release syndrome. The information material should indicate that allergic reactions may exhibit only as mild pruritic rashes or they may be severe such as erythroderma, Stevens-Johnson syndrome, vasculitis, or anaphylaxis. The information material should also indicate that anaphylactic reactions (anaphylaxis) are serious and occasionally fatal hypersensitivity reactions. Allergic reactions including anaphylaxis may occur when any foreign protein is injected into the body. They may range from mild manifestations such as urticaria or rash to lethal systemic reactions. Anaphylactic reactions occur soon after exposure, usually within 10 minutes. Patients may experience paresthesia, hypotension, laryngeal oedema, mental status changes, facial or pharyngeal angioedema, airway obstruction, bronchospasm, urticaria and pruritus, serum sickness, arthritis, allergic nephritis, glomerulonephritis, temporal arthritis, or eosinophilia.

### Kits

The present invention also provides a pharmaceutical pack or kit comprising one or more containers filled with reagents for detecting, monitoring and/or measuring embryonic stem cells associated with fibrotic lesions. In one example, the pharmaceutical pack or kit optionally comprises instructions for the use of the reagents provided for detecting and/or measuring embryonic stem cells associated with fibrotic lesions. In another example, the pharmaceutical pack or kit optionally comprises a notice in the form prescribed by a governmental agency regulating the manufacture, use or sale of pharmaceuticals or biological products, which notice reflects approval by the agency of manufacture, for use or sale for human administration.

In an example, the pharmaceutical pack or kit comprises in one or more containers a embryonic stem cell surface marker-binding agent. In certain examples, the agent is an antibody that selectively or specifically binds to an embryonic stem cell surface marker, wherein the embryonic stem cell is associated with a fibrotic lesion. The agent may be an antibody (including, e.g., human, humanised, chimeric, monoclonal, polyclonal, Fvs, ScFvs, Fab or F(ab)2 fragments or epitope binding fragments), which cross-reacts with any embryonic stem cell surface marker. In accordance with this example, the pharmaceutical pack or kit comprises one or more antibodies which bind to an embryonic stem cell surface marker, wherein each antibody binds to a different epitope of the embryonic stem cell surface marker and/or binds to the embryonic stem cell surface marker with a different affinity.

For antibody based kits, the kit can comprise, for example: (1) a first antibody (which may or may not be attached to a solid support) which binds to a embryonic stem cell surface marker protein; and, optionally, (2) a second, different antibody which binds to either the embryonic stem cell surface marker protein bound by the first antibody, or the first antibody and is conjugated to a detectable label (e.g., a fluorescent label, radioactive isotope or enzyme). The antibody-based kits may also comprise beads for conducting an immunoprecipitation. Each component of the antibody-based kits is generally in its own suitable container. Thus, these kits generally comprise distinct containers suitable for each antibody. Further, the antibody-based kits may comprise instructions for performing the assay and methods for interpreting and analysing the data resulting from the performance of the assay.

For nucleic acid micoarray kits, the kits generally comprise (but are not limited to) probes specific for certain genes attached to a solid support surface. In other examples, the probes are soluble. In one such example, probes can be either oligonucleotides or longer length probes including probes ranging from 150 nucleotides in length to 800 nucleotides in length. The probes may be labelled with a detectable label. The microarray kits may comprise instructions for performing the assay and methods for interpreting and analysing the data resulting from the performance of the assay. The kits may also comprise hybridisation reagents and/or reagents necessary for detecting a signal produced when a probe hybridises to a stem cell surface marker nucleic acid sequence. Generally, the materials and reagents for the microarray kits are in one or more containers. Each component of the kit is generally in its own suitable container.

For Quantitative RT-PCR, the kits generally comprise pre-selected primers specific for certain embryonic stem cell surface marker nucleic acid sequences. The Quantitative RT-PCR kits may also comprise enzymes suitable for amplifying nucleic acids (e.g., polymerases such as Taq), and deoxyribonucleotides and buffers needed for the reaction mixture for amplification. The Quantitative RT-PCR kits may also comprise probes specific for the nucleic acid sequences associated with or indicative of a condition. The probes may or may not be labelled with a flourophore. The probes may or may not be labelled with a quencher molecule. In some examples, the Quantitative RT-PCR kits also comprise components suitable for reverse- transcribing RNA including enzymes (e.g., reverse transcriptases such as AMV, MMLV and the like) and primers for reverse transcription along with deoxynucleotides and buffers needed for the reverse transcription reaction. Each component of the quantitative RT-PCR kit is generally in its own suitable container. Thus, these kits generally comprise distinct containers suitable for each individual reagent, enzyme, primer and probe. Further, the quantitative RT-PCR kits may comprise instructions for performing the assay and methods for interpreting and analysing the data resulting from the performance of the assay.

A kit can optionally further comprise a predetermined amount of an isolated stem cell surface marker polypeptide or a nucleic acid encoding a stem cell surface marker, e.g., for use as a standard or control. The diagnostic methods of the present invention can assist in conducting or monitoring a clinical study. In accordance with the present invention, suitable test samples, e.g., of serum or tissue, obtained from a subject can be used for diagnosis.

Based on the results obtained by use of the pharmaceutical pack or kit (i.e. whether the stem cell amount has stabilised or decreased), the medical practitioner administering the therapy or regimen may choose to continue the therapy or regimen. Alternatively, based on the result that the stem cell amount has increased, the medical practitioner may choose to continue, alter or halt the therapy or regimen.

Any reference to prior art documents in this specification is not to be considered an admission that such prior art is widely known or forms part of the common general knowledge in the field.

As used in this specification, the words “comprises”, “comprising”, and similar words, are not to be interpreted in an exclusive or exhaustive sense. In other words, they are intended to mean “including, but not limited to”.

The invention is further described with reference to the following examples. It will be appreciated that the invention as claimed is not intended to be limited in any way by these examples.

### EXAMPLES

### Example 1: Materials and Methods

### Tissue Samples and Patient Characteristics

Formalin-fixed, paraffin-embedded keloid scar samples with matching snap-frozen tissues obtained from 3 female and 3 male patients, with a mean age of 29 (range: 22 to 55) years, were used in this study.

### Immunohistochemistry

Immunohistochemical (IHC) staining was performed on 5 μm-thick formalin-fixed paraffin-embedded sections of keloid scar samples from 6 patients, according to a protocol approved by the Central Regional Health and Disability Ethics Committee. Antigen retrieval was performed using sodium citrate (Leica, Sydney, Australia) at 95° C. for 15 min, followed by 3,3 diaminobenzidine (DAB) staining with primary antibodies: SOX2, 1:500 (Thermofisher Scientific, CA, USA); pSTAT3, 1:100 (Cell Signalling Technology, MA, USA); OCT4, 1:30 (Cell Marque, CA, USA); NANOG, 1:1000 (Cell Signalling Technology); von Willebrand factor (vWF); 1:200 (Dako, Glostrup, Denmark); tryptase; ready-to-use (Leica) using the bond polymer refine detection kit (Leica). To confirm dual expression of the proteins, representative sections of keloid scar were used for immunofluorescent (IF) IHC staining with the same primary antibodies at the same concentrations, with an appropriate secondary antibody for detection (donkey anti-mouse Alexa-488 or donkey anti-rabbit Alexa 594 (Life Technologies, Carlsbad, Calif., USA).

All antibodies were diluted in Bond primary diluent (Leica), and all DAB IHC and IF IHC staining were performed on the Leica Bond Rx auto-stainer (Leica). The DAB IHC and IF IHC stained slides were mounted using either Surgipath Micromount mounting medium (Leica) or Vecta Shield Hardset mounting medium with 4′,6′ diamidino-2-phenylindole (Vector Laboratories, CA, USA), respectively. Human seminoma and tonsillar tissue sections were used as positive controls for the ESC markers (SOX2, pSTAT, OCT4 and NANOG) and the lymphocyte markers (CD20 and CD3), respectively.

### NanoString Analysis

Snap-frozen keloid scar samples from the original cohort of 6 patients were used to isolate total RNA for NanoString nCounter Gene Expression Assay (NanoString Technologies, Seattle, Wash., USA). RNA was extracted from snap-frozen keloid scar tissues using RNeasy Mini Kit (Qiagen), and quantitated by the NanoDrop 2000 Spectrophotometer (Thermo Scientific). RNA was subjected to the NanoString nCounter™ gene expression assay as completed by New Zealand Genomics Ltd (Dunedin, NZ), according to the manufacturer's protocol. Probes for the genes encoding for OCT4 (NM_002701.4), NANOG (NM_024865.2), STAT3 (NM_139276.2) and the housekeeping gene GUSB (NM_00181.3) were designed and synthesised by NanoString Technologies. Raw data was analysed by nSolver software (NanoString Technologies) using standard settings and were normalised against the housekeeping gene.

### In-situ Hybridisation

4 μm-thick formalin-fixed paraffin-embedded keloid tissue sections (n=3) from the original cohort of 6 patients were used for mRNA in-situ hybridisation (ISH) staining, on the Leica Bond Rx autostainer and detected using the ViewRNA red stain kit (Affymetrix, CA, USA). OCT4 (NM_002701.4), NANOG (NM_024865.2), SOX2 (NM_003106) and STAT3 (NM_139276.2) probes were obtained from Affymetrix. Human seminoma tissue sections were used as positive controls for these ESC markers.

### Image Analysis

Confocal microscopy images were captured using the Olympus FV1200 confocal microscope (Tokyo, Japan). Bright field images were captured using the Olympus BX53 light microscope fitted with an Olympus DP21 digital camera (Tokyo, Japan).

### Example 2: Results

### Immunohistochemical Staining

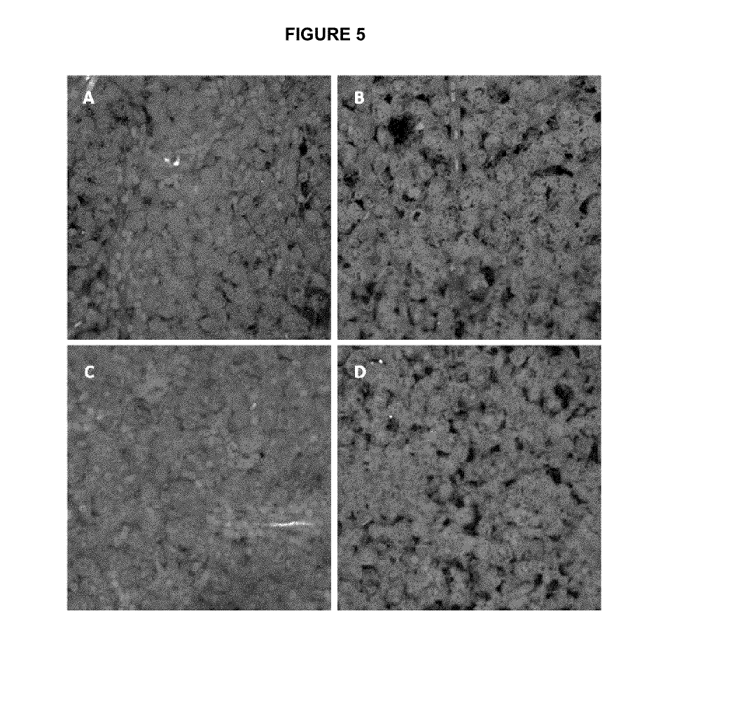

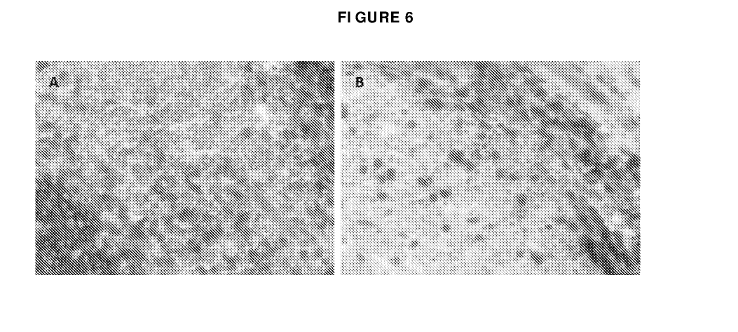

To investigate the expression of ESC markers in keloid scar Applicants initially examined the expression of OCT4 (FIG. 1A, green), which was localised to the endothelium of the microvessels that expressed vWF (FIG. 1A, red). To further characterise this ESC population, Applicants examined the expression of SOX2 (FIG. 1B, red), involved in ESC gene support (Itinteang, 2012), which was also expressed on the CD34⁺ endothelium (FIG. 1B, green). pSTAT3 (FIG. 1C, red), the activated form of STAT3, has been associated with ESC self-renewal (Niwa, 1998), was also expressed on the CD34⁺ endothelium (FIG. 1C, green). NANOG (FIG. 1D, red), another ESC marker which is associated with ESC pluripotency (Loh, 2006), was also expressed on the CD34⁺ endothelium (FIG. 1D, green). The abundance of nucleated cells (FIG. 1A-D) immediately adjacent to the endothelium that expressed these ESC markers, appeared similar to a recent report of KALTs in keloid scar and led Applicants to investigate the expression of the KALT-associated markers (Bagabir, 2012) CD20 (FIG. 2A, brown) and CD3 (FIG. 2B, brown) in the same keloid scar samples. Staining of the same phenotypic endothelium, characterised by the expression of vWF (FIG. 2 A & B, red) confirmed the expression of these ESC markers in the endothelium of the KALTs. Human seminoma and tonsillar controls confirmed the specificity of the ESC markers and the lymphoid markers (FIGS. 5 and 6).

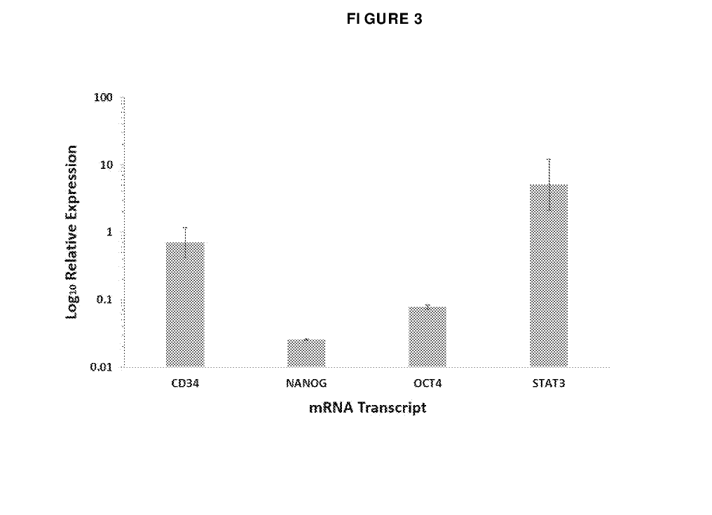

NanoString Analysis Transcriptional profiling confirmed the relative abundance of CD34, OCT4, SOX2, STAT3, and NANOG in keloid scar tissue relative to the housekeeping gene, HPRT1 (FIG. 3).

### In-situ Hybridisation

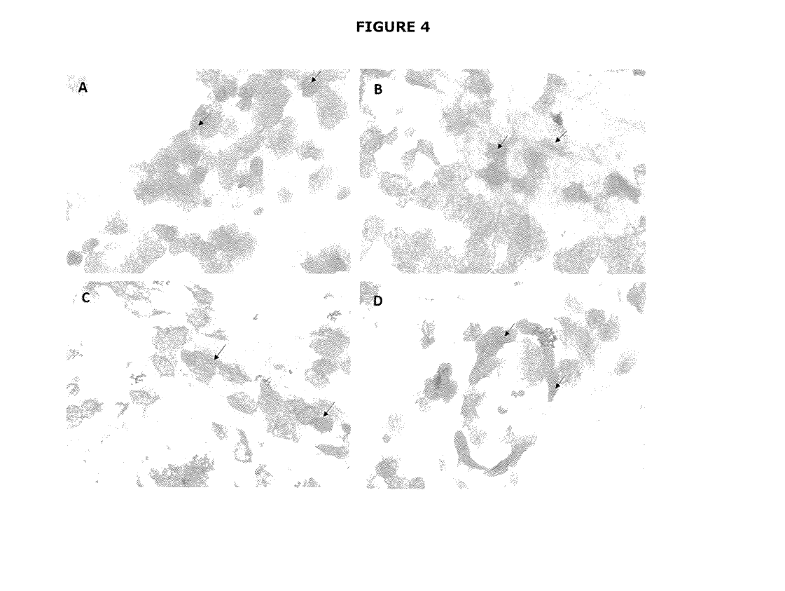

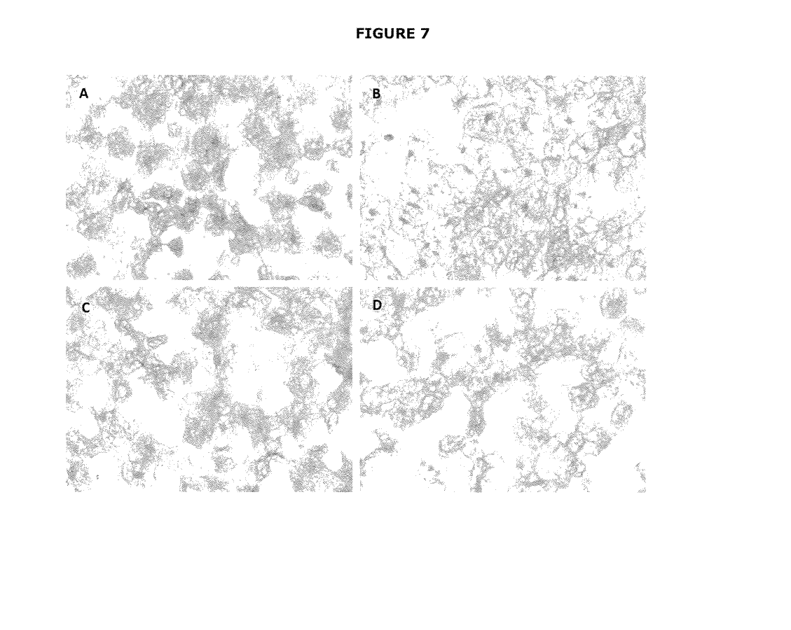

ISH staining confirmed the mRNA expression of OCT4 (FIG. 4A, red, arrows), SOX2 (FIG. 4B, red, arrows), STAT3 (FIG. 4C, red, arrows), and NANOG (FIG. 4D, red, arrows) in keloid scar tissue. The specificity of the ISH probes was confirmed with human seminoma which were used as positive controls (FIG. 7).

### Example 3: Discussion

The presence of T cells, B cells, mast cells and M2 macrophages has been recently demonstrated in keloid scars and has been referred to as KALTs (Bagabir, 2012). One report describes the effect of IL6/IL17 in the development of keloid scar (Zhang, 2009).

Applicant's results highlight the intriguing novel finding of the expression of the ESC markers OCT4, SOX2, NANOG and pSTAT3 on the endothelium of the microvessels surrounded by the KALTs which are located just beneath the epidermis (Bagabir, 2012).

It is exciting to speculate that this primitive endothelium within keloid scar may potentially be the source of both myofibroblasts and KALTs, although this remains to be conclusively determined.

ESCs are undifferentiated cells possessing the ability to differentiate and proliferate indefinitely (Bishop, 2002). They may be induced to differentiate into a broad range of cell types, with vastly different properties (Bishop, 2002). The fate of stem cells within tissues is determined by the micro-environmental niche (Li, 2006). Normally proliferation of embryonic-like stem cell is under tight control, with balanced proliferating-promoting and proliferation-inhibiting stimuli from the microenvironment (Moon, 2008). Loss of this controlled niche typically leads to embryonic-like stem cell depletion (Scadden, 2006). This suggests that in tissues such as keloid scar, there is a putative trigger that potentially allows both the upregulation of embryonic-like stem cell markers as well as the ability of the cells to differentiate and form KS.

The similarities between KS mesenchymal stem cells and skin-derived precursors suggest that they are potentially derived from neural crest precursors which may persist at low levels within normal adult skin (Choi, 2008). Inflammatory pathways within the keloid scar microenvironment have been shown to increase the number of benign tumour-like stem cells (Zhang, 2009). Given that the KALTs influence inflammatory stimuli within keloid scar, it is exciting to speculate that an alteration of the stem cell niche induces the microenvironmental changes conducive for the development of keloid scar.

This is the first report demonstrating the expression of an embryonic-like stem cell population within the increased concentration of lymphoid tissue aggregates within keloid scar. This study infers that the presence of a phenotypically primitive endothelium is surrounded by or is in the vicinity of the KALTs. More intriguingly is the role of the KALTs in the maintenance of this primitive stem cell population that may underscore the development of keloid scar.

The identification of a primitive stem cell population within keloid scar located just beneath the epidermal layer, adjacent to KALTs, offers novel insights into keloid scar, and presents a potential novel target for the treatment of keloid scar.

Although the invention has been described by way of example, it should be appreciated that variations and modifications may be made without departing from the scope of the invention as defined in the claims. Furthermore, where known equivalents exist to specific features, such equivalents are incorporated as if specifically referred in this specification.

### REFERENCES

1. Thompson L D R. Skin keloid. Ear, Nose, & Throat Journal 2004;83(8):519
2. Berman B, Bieley H C. Keloids. Journal of the American Academy of Dermatology 1995;33(1):117-23
3. Muir I F K. On the nature of keloid and hypertrophic scars. British Journal of Plastic Surgery 1990;43(1):61-69 doi: 10.1016/0007-1226(90)90046-3[published Online First: Epub Date]|.
4. English R S, Shenefelt P D. Keloids and Hypertrophic Scars. Dermatologic Surgery 1999;25(8):631-38
5. Al-Attar A, Mess S, Thomassen J M, Kauffman C L, Davison S P. Keloid pathogenesis and treatment. Plastic and reconstructive surgery 2006;117(1):286-300
6. Furtado F, Hochman B, Ferreira L. Evaluating keloid recurrence after surgical excision with prospective longitudinal scar assessment scales. Journal of Plastic, Reconstructive & Aesthetic Surgery 2012;65(7):e175-e81
7. Botwood N, Lewanski C, Lowdell C. The risks of treating keloids with radiotherapy. The British Journal of Radiology 1999;72(864):1222-24
8. James W D, Besanceney C D, Odom R B. The ultrastructure of a keloid. Journal of the American Academy of Dermatology 1980;3(1):50-57 doi: 10.1016/S0190-9622(80)80224-6[published Online First: Epub Date]|.
9. Zhang Q, Yamaza T, Kelly A P, et al. Tumor-Like Stem Cells Derived from Human Keloid Are Governed by the Inflammatory Niche Driven by IL-17/IL-6 Axis (Niche-Driven Overgrowth of KPC). PLoS ONE 2009;4(11):1-16 doi: 10.1371/journal.pone.0007798[published Online First: Epub Date]|.
10. Bagabir R, Byers R J, Chaudhry I H, Müller W, Paus R, Bayat A. Site-specific immunophenotyping of keloid disease demonstrates immune upregulation and the presence of lymphoid aggregates. The British Journal of Dermatology 2012;167(5):1053-66
11. Itinteang T, Tan S T, Brasch H D, et al. Infantile haemangioma expresses embryonic stem cell markers. Journal of Clinical Pathology 2012:jclinpath-2011-200462
12. Niwa H, Burdon T, Chambers I, Smith A. Self-renewal of pluripotent embryonic stem cells is mediated via activation of STAT3. Genes & Development 1998;12(13):2048-60
13. Loh Y-H, Wu Q, Chew J-L, et al. The Oct4 and Nanog transcription network regulates pluripotency in mouse embryonic stem cells. Nature Genetics 2006;38(4):431-40
14. Bishop A E, Buttery L D, Polak J M. Embryonic stem cells. The Journal of pathology 2002;197(4):424-29
15. Li L, Neaves W B. Normal stem cells and cancer stem cells: the niche matters. Cancer Research 2006;66(9):4553-57
16. Moon J-H, Kwak S S, Park G, et al. Isolation and characterization of multipotent human keloid-derived mesenchymal-like stem cells. Stem Cells and Development 2008;17(4):713-24
17. Scadden D T. The stem-cell niche as an entity of action. Nature 2006;441(7097):1075-79
18.Choi S-C, Jun E K, Jung H-Y, et al. Isolation and characterization of multipotent human keloid-derived mesenchymal-like stem cells. Stem Cells and Development 2008;17:713

1. A method for preventing, treating, or managing a fibrotic condition in a patient in need thereof, the method comprising administering one or more therapeutic agents to the patient in an amount sufficient to selectively eradicate, or inhibit the growth, proliferation and/or differentiation of embryonic stem cells associated with a fibrotic lesion, wherein the embryonic stem cells are characterised by (i) the expression of one or more embryonic stem cell biomarkers, and (ii) the expression of one or more biomarkers associated with the Renin-Angiotensin System.

2. The method according to claim 1, wherein the embryonic stem cells are characterised by expression of one or more embryonic stem cell biomarkers selected from the group consisting of Cripto, ABCG2, Alkaline Phosphatase/ALPL, CD9, FGF-4, GDF-3, Integrin alpha 6/CD49f, Integrin beta 1/CD29, NANOG, OCT3/4, Podocalyxin, SOX2, SSEA-3, SSEA-4, STAT3, SSEA-1, FoxD3, DPPAS/ESG1, Rex-1/ZFP42, DPPA4, LIN-28A, UTF1, Lefty-A, Lefty-1, TBX3, ESGP, TRA-1-60(R), TRA-1-81, 5T4, TBX2, ZIC3, CD30/TNFRSF8, KLFS, c-Myc, GCNF/NR6A1, SUZ12, Smad2, CDX2, TROP-2, CD117/c-kit, LIN-41, Integrin alpha 6 beta 4, THAP11, Smad2/3, TBXS, TEX19, OCT4A, TEX19.1, DPPA2, Activin RIB/ALK-4, Activin RIIB, FGF-5, GBX2, Stella/Dppa3, DNMT3B, F-box protein 15/FBXO15, LIN-28B, Integrin alpha 6 beta 1, KLF4, ERR beta/NR3B2, EpCAM/TROP1, TERT, CHD1, Cbx2, c-Maf and L1TD1.

3. The method according to claim 1, wherein the embryonic stem cells are characterised by expression of one or more embryonic stem cell biomarkers selected from the group consisting of OCT4, SOX2, NANOG and PSTAT3.

4. The method according to claim 1, wherein the embryonic stem cells are characterised by expression of the embryonic stem cell biomarkers OCT4, SOX2, NANOG and PSTAT3.

5. The method according to claim 1, wherein the embryonic stem cells are characterised by expression of one or more Renin-Angiotensin System biomarkers selected from the group consisting of Renin Receptor, Angiotensin Converting Enzyme, Angiotensin II Receptor 1, angiotensin II receptor 2, a soluble form of the Renin Receptor and a soluble form of Angiotensin Converting Enzyme.

6. The method according to claim 1, wherein the fibrotic condition is selected from the group consisting of liver fibrosis, kidney fibrosis, lung fibrosis, hypertrophic scars, keloid scar, dupuytren's contracture and desmoid tumours.

7. The method according to claim 6, wherein the fibrotic condition is keloid scar.

8. The method according to claim 1, wherein the therapeutic agent is selected from the group consisting of Direct Renin Inhibitors (DRIs), Angiotensin-Converting Enzyme Inhibitors (ACEIs), Angiotensin Receptor Blockers (ARBs), Beta-Blockers, Cyclo-oxygenase 2 Inhibitors, Chymase Inhibitors, Inhibitors of Cathepsin B, Cathepsin D and Cathepsin G, Calcium, Vitamin D, and Calcium Channel Blockers.

9. A method for preventing, treating, or managing a fibrotic condition in a patient in need thereof, the method comprising administering a therapeutic agent to the patient in an amount sufficient to selectively eradicate or inhibit the growth, proliferation and/or differentiation of embryonic stem cells associated with a fibrotic lesion, wherein the embryonic stem cells are characterised by (i) expression of the embryonic stem cell biomarkers OCT4, SOX2, NANOG and PSTAT3, and (ii) expression of the Renin-Angiotensin System biomarkers Renin Receptor and Angiotensin II Receptor 2, and wherein the therapeutic agent in selected from the group consisting of Direct Renin Inhibitors (DRIs), Angiotensin-Converting Enzyme Inhibitors (ACEIs), Angiotensin Receptor Blockers (ARBs), Beta-Blockers, Cyclo-oxygenase 2 Inhibitors, Chymase Inhibitors, Inhibitors of Cathepsin B, Cathepsin D and Cathepsin G, Calcium, Vitamin D, and Calcium Channel Blockers.

10. A method for determining presence or absence of a fibrotic condition in a subject, the method comprising:
(i) detecting and/or measuring the levels of embryonic stem cells present in a biological sample obtained from the subject using biomarker expression analysis;
(ii) comparing the levels of the embryonic stem cells obtained from the biological sample against the embryonic stem cell level from a control population;
wherein, an increased level in the embryonic stem cells obtained from the biological sample relative to the control population is diagnostic that the subject has, or is predisposed to developing, a fibrotic lesion.

11. The method according to claim 10, further comprising the step of:
(iii) administering a prophylactic or therapeutic regime to the subject who has, or is predisposed to developing, a fibrotic lesion.

12. A pharmaceutical composition for use in a method for treatment of a fibrotic condition, wherein the pharmaceutical composition comprises a therapeutic agent(s) sufficient to selectively eradicate or, inhibit the growth, proliferation and/or differentiation of embryonic stem cells associated with a fibrotic lesion, and wherein the method comprises administering the therapeutic agent to a patient with a fibrotic condition.

13. The pharmaceutical composition according to claim 12, wherein the therapeutic agent is selected from the group consisting of Direct Renin Inhibitors (DRIs), Angiotensin-Converting Enzyme Inhibitors (ACEIs), Angiotensin Receptor Blockers (ARBs), Beta-Blockers, Cyclo-oxygenase 2 Inhibitors, Chymase Inhibitors, Inhibitors of Cathepsin B, Cathepsin D and Cathepsin G, Calcium, Vitamin D, and Calcium Channel Blockers.

14. A kit for use in the treatment of a fibrotic condition, the kit comprising a therapeutic agent sufficient to selectively eradicate, or inhibit the growth, proliferation and/or differentiation of embryonic stem cells associated with a fibrotic lesion, together with instructions for how to administer a therapeutic dose to the subject.

15. The kit according to claim 14, wherein the therapeutic agent is selected from the group consisting of Direct Renin Inhibitors (DRIs), Angiotensin-Converting Enzyme Inhibitors (ACEIs), Angiotensin Receptor Blockers (ARBs), Beta-Blockers, Cyclo-oxygenase 2 Inhibitors, Chymase Inhibitors, Inhibitors of Cathepsin B, Cathepsin D and Cathepsin G, Calcium, Vitamin D, and Calcium Channel Blockers.
